(19) United States
(12) Patent Application Publication
(10) Pub. No.: US 2025/0111936 A1
(43) Pub. Date: Apr. 3, 2025

(54) SYSTEMS AND METHODS FOR PREDICTING RESOURCE SYSTEM RE-UTILIZATION

(71) Applicant: Optum, Inc., Minnetonka, MN (US)

(72) Inventors: Payal THAPAR, Noida (IN); Biren RAJDEV, Phillaur (IN); Ankit KINDRA, Delhi (IN); Sanket BHARADWAJ, Noida (IN); Saurabh BHARGAVA, Eden Prairie, MN (US); Vibhor DAWAR, Gurgaon (IN)

(21) Appl. No.: 18/680,413

(22) Filed: May 31, 2024

Related U.S. Application Data
(60) Provisional application No. 63/586,196, filed on Sep. 28, 2023.

(51) Int. Cl. G16H 40/20; G16H 50/70

(52) U.S. Cl. CPC G16H 40/20; G16H 50/70

(57) ABSTRACT

A method includes: receiving a first set of data associated with an element during a first stage of a plurality of stages; applying a first stage machine learning model to the first set of data to generate a prediction value, wherein the first stage machine learning model is trained with a first set of feature data that is available during the first stage; updating the prediction value by: receiving a second set of data associated with the element during a second stage of the plurality of stages; and applying a second stage machine learning model to the second set of data, wherein the second stage machine learning model is trained with (i) the first set of feature data, and (ii) a second set of feature data that is available during the second stage; and initiating performance of a mitigation action based on the updated prediction value.

### CROSS-REFERENCE TO RELATED APPLICATION(S)

This application claims the benefit of priority to U.S. Provisional Patent Application No. 63/586,196, filed Sep. 28, 2023, the entirety of which is incorporated by reference herein.

### TECHNICAL FIELD

The present disclosure generally relates to the field of data analytics and artificial intelligence. Particularly, the present disclosure relates to systems and methods for providing an identification framework to determine a risk of a subsequent re-utilization of a resource system.

### BACKGROUND

An entity may be released from a resource system, and may subsequently be readmitted to the resource system. A likelihood of readmission to the resource system may increase or decrease based on a quality of care provided to the entity or an adherence by the entity to a care plan, for example. Readmission to the resource system may be undesirable for the entity and may require additional support from the resource system. The inability to accurately determine a risk of readmission for an entity may preclude mitigation of various contributing factors that may lead to readmission of the entity to the resource system.

Conventional techniques for predicting and mitigating preventable resource utilization due to readmission of an entity to a resource system typically use datasets available at a specific point in time and, as a result, the data analysis and prediction conclude either prematurely without considering what may take place in the near future or too late after failing to detect preventable readmission risks earlier in time. The conventional techniques thus have several drawbacks. For example, these techniques fail to consider that the entity's need for readmission may change over time and under varying circumstances. As a result, the techniques often rely on a single model or algorithm to make a prediction for an entity at a specific point in time, and only a limited amount of data is collected and analyzed to make such a prediction.

The present disclosure is directed to overcoming the above-mentioned challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

In some aspects, the techniques described herein relate to a computer-implemented method including: receiving, by one or more processors, a first set of data associated with an element during a first stage of a plurality of stages; applying, by the one or more processors, a first stage machine learning model to the first set of data to generate a prediction value, wherein the first stage machine learning model is trained with a first set of feature data that is available during the first stage; updating, by the one or more processors, the prediction value by: receiving a second set of data associated with the element during a second stage of the plurality of stages; and applying a second stage machine learning model to the second set of data, wherein the second stage machine learning model is trained with (i) the first set of feature data that is available during the first stage, and (ii) a second set of feature data that is available during the second stage; and initiating, by the one or more processors, performance of a mitigation action based on the updated prediction value.

In some aspects, the techniques described herein relate to a computer-implemented method, further including: training one or more of the first stage machine learning model or the second stage machine learning model by: receiving first data regarding an element attribute; extracting a first feature from the received first data; receiving second data regarding a training prediction value related to the element attribute; extracting a second feature from the received second data; and training the one or more of the first stage machine learning model or the second stage machine learning model to learn an association between the element attribute and the training prediction value related to the element attribute, based on the extracted first feature and the extracted second feature.

In some aspects, the techniques described herein relate to a computer-implemented method, wherein one or more of the first stage machine learning model or the second stage machine learning model includes one or more of a neural network, regression, random forest, or gradient boosting.

In some aspects, the techniques described herein relate to a computer-implemented method, wherein the updating, by the one or more processors, the prediction value further includes: receiving a third set of data associated with the element during a third stage of the plurality of stages; and applying a third stage machine learning model to the third set of data, wherein the third stage machine learning model is trained with (i) the first set of feature data that is available during the first stage, (ii) the second set of feature data that is available during the second stage, and (iii) a third set of feature data that is available during the third stage.

In some aspects, the techniques described herein relate to a computer-implemented method, wherein the updating, by the one or more processors, the prediction value further includes: receiving a fourth set of data associated with the element during a fourth stage of the plurality of stages; and applying a fourth stage machine learning model to the fourth set of data, wherein the fourth stage machine learning model is trained with (i) the first set of feature data that is available during the first stage, (ii) the second set of feature data that is available during the second stage, (iii) the third set of feature data that is available during the third stage, and (iv) a fourth set of feature data that is available during the fourth stage.

In some aspects, the techniques described herein relate to a computer-implemented method, wherein: the first stage is a stage of initial utilization of a resource system by the element, the second stage is a stage during a stay of the element in the resource system, the third stage is a stage of discharge of the element from the resource system, and the fourth stage is a stage after discharge of the element from the resource system.

In some aspects, the techniques described herein relate to a computer-implemented method, wherein the first set, historical prior authorizations, clinical and/or electronic medical records, distance between resource system and element, chronic condition details of the element, risk scores for each resource system, diagnostic code level, historical hospitalization-related features, prescription-related features, lifestyle features, or social determinants of health.

In some aspects, the techniques described herein relate to a computer-implemented method, wherein the second set of data includes one or more of live availability of beds, resource system burnout, partial in hospital treatment, or resource system and facility related features of the element.

In some aspects, the techniques described herein relate to a computer-implemented method, wherein the third set of data includes one or more of facility treatment, resource system information, facility information, or a discharge plan.

In some aspects, the techniques described herein relate to a computer-implemented method, wherein the fourth set, prior authorizations, resource system and/or facility visits, call comments, vitals of the element, or information related to a smart device of the element.

In some aspects, the techniques described herein relate to a computer-implemented method, wherein the mitigation action includes one or more of updating a care plan for the element in a resource system, addressing a gap in care for the element in the resource system, generating a follow-up for the element, updating a discharge plan for the element, or contacting the element.

In some aspects, the techniques described herein relate to a computer-implemented method, wherein the updating, by the one or more processors, the prediction value is performed periodically.

In some aspects, the techniques described herein relate to a computer-implemented method, wherein the prediction value represents a likelihood of re-utilization of a resource system by the element.

In some aspects, the techniques described herein relate to a computer-implemented method, wherein the initiating, by the one or more processors, the performance of the mitigation action includes generating a display including one or more of the mitigation action or the updated prediction value.

In some aspects, the techniques described herein relate to a system including: one or more processors; and one or more memories storing instructions that, when executed by the one or more processors, cause the one or more processors to perform operations including: receiving a first set of data associated with an element during a first stage of a plurality of stages; applying a first stage machine learning model to the first set of data to generate a prediction value, wherein the first stage machine learning model is trained with a first set of feature data that is available during the first stage; updating the prediction value by: receiving a second set of data associated with the element during a second stage of the plurality of stages; and applying a second stage machine learning model to the second set of data, wherein the second stage machine learning model is trained with (i) the first set of feature data that is available during the first stage, and (ii) a second set of feature data that is available during the second stage; and initiating performance of a mitigation action based on the updated prediction value.

In some aspects, the techniques described herein relate to a system, wherein the operations further include: training one or more of the first stage machine learning model or the second stage machine learning model by: receiving first data regarding an element attribute; extracting a first feature from the received first data; receiving second data regarding a training prediction value related to the element attribute; extracting a second feature from the received second data; and training the one or more of the first stage machine learning model or the second stage machine learning model to learn an association between the element attribute and the training prediction value related to the element attribute, based on the extracted first feature and the extracted second feature.

In some aspects, the techniques described herein relate to a system, wherein one or more of the first stage machine learning model or the second stage machine learning model includes one or more of a neural network, regression, random forest, or gradient boosting.

In some aspects, the techniques described herein relate to a system, wherein the updating the prediction value further includes: receiving a third set of data associated with the element during a third stage of the plurality of stages; applying a third stage machine learning model to the third set of data, wherein the third stage machine learning model is trained with (i) the first set of feature data that is available during the first stage, (ii) the second set of feature data that is available during the second stage, and (iii) a third set of feature data that is available during the third stage; receiving a fourth set of data associated with the element during a fourth stage of the plurality of stages; and applying a fourth stage machine learning model to the fourth set of data, wherein the fourth stage machine learning model is trained with (i) the first set of feature data that is available during the first stage, (ii) the second set of feature data that is available during the second stage, (iii) the third set of feature data that is available during the third stage, and (iv) a fourth set of feature data that is available during the fourth stage.

In some aspects, the techniques described herein relate to a system, wherein: the first stage is a stage of initial utilization of a resource system by the element, the second stage is a stage during a stay of the element in the resource system, the third stage is a stage of discharge of the element from the resource system, and the fourth stage is a stage after discharge of the element from the resource system.

In some aspects, the techniques described herein relate to a non-transitory computer readable medium storing instructions that, when executed by one or more processors, cause the one or more processors to perform operations including: receiving a first set of data associated with an element during a first stage of a plurality of stages; applying a first stage machine learning model to the first set of data to generate a prediction value, wherein the first stage machine learning model is trained with a first set of feature data that is available during the first stage; updating the prediction value by: receiving a second set of data associated with the element during a second stage of the plurality of stages; and applying a second stage machine learning model to the second set of data, wherein the second stage machine learning model is trained with (i) the first set of feature data that is available during the first stage, and (ii) a second set of feature data that is available during the second stage; and initiating performance of a mitigation action based on the updated prediction value.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are explanatory only and are not restrictive of the disclosed embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1 depicts an example system infrastructure for a re-utilization prediction platform, according to one or more embodiments.

FIG. 2 depicts an example flowchart of a re-utilization prediction, according to one or more embodiments.

FIG. 3 depicts an example timeline of utilization of a resource system and corresponding re-utilization predictions, according to one or more embodiments.

FIG. 4A and FIG. 4B depict an example system for re-utilization prediction using four trained machine-learning based models, according to one or more embodiments.

FIG. 5 depicts an example system infrastructure for a re-utilization prediction platform, according to one or more embodiments.

FIG. 6 depicts a flowchart of a method of re-utilization prediction using two trained machine-learning based models, according to one or more embodiments.

FIG. 7 depicts a flowchart of a method of training a machine-learning based model to determine a re-utilization prediction, according to one or more embodiments.

FIG. 8 depicts an implementation of a computer system that executes techniques presented herein, according to one or more embodiments.

FIG. 9 depicts a flow diagram for training a machine learning model, according to one or more embodiments.

### DETAILED DESCRIPTION OF EMBODIMENTS

The present disclosure generally relates to the field of data analytics and artificial intelligence. Particularly, the present disclosure relates to systems and methods for providing an identification framework to determine a risk of a subsequent re-utilization of a resource system.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section.

An entity may be released from a resource system, and may subsequently be readmitted to the resource system. A likelihood of readmission to the resource system may increase or decrease based on a quality of care provided to the entity or an adherence by the entity to a care plan, for example. Readmission to the resource system may be undesirable for the entity and may require additional support from the resource system. The inability to accurately determine a risk of readmission for an entity may preclude mitigation of various contributing factors that may lead to readmission of the entity to the resource system.

Conventional solutions determine a risk of a subsequent re-utilization of a resource system at a single point in time, such as at a time of release of the entity from the resource system. However, such a determination at a time of release of the entity from the resource system fails to address an issue related to the entity while the entity is still utilizing the resource system and not released.

One or more embodiments of the current disclosure provide a technical improvement over conventional techniques by using a non-conventional multi-stage approach that updates a prediction score of a subsequent re-utilization of a resource system as more data becomes available as the entity progresses through different stages during a multi-stage health care journey, in order to advantageously make more accurate predictions than conventional techniques using the non-conventional arrangement of multiple models corresponding to the respective stages.

One or more embodiments of the current disclosure determine entities with a high risk of a subsequent re-utilization of a resource system (e.g., high-risk entities) at multiple points in time (e.g., four different stages of utilization of the resource system by the entity). This multi-stage approach provides technical improvements and advantages over conventional methodologies, namely a timely prediction of subsequent re-utilization risk, thus enabling proactive mitigation actions prior to a time of release of the entity from the resource system. The multi-stage approach thus allows for additional opportunities for performing mitigation actions, by determining high risk entities from initial utilization by the entity of the resource system to a time following a release of the entity from the resource system to reduce the risk of a subsequent re-utilization of the resource system.

The risk reduction resulting from the performance of the mitigation actions further leads to a significant saving of computational as well as other resources in the resource systems, and mitigates system bottlenecks within and between the resource systems. Furthermore, mitigation actions are identified and provided automatically and expeditiously based on the results of, and datasets used in, the risk determination, further contributing to the resource savings and expediting the performance of the mitigation actions. Also, the mitigation action effects a particular treatment or prophylaxis for a disease or medical condition.

One or more embodiments initiate proactive performance of mitigation actions based on the risk determined at each stage of utilization of the resource system by the entity. For example, a resource utilization plan including one or more mitigation actions is generated and/or updated during utilization of the resource system by the entity, a release plan including one or more mitigation actions is generated at release of the entity from the resource system, and the release plan is modified during a post-release stage of the entity from the resource system. In some embodiments, a resource system performance with respect to subsequent re-utilization is determined for one or more resource systems.

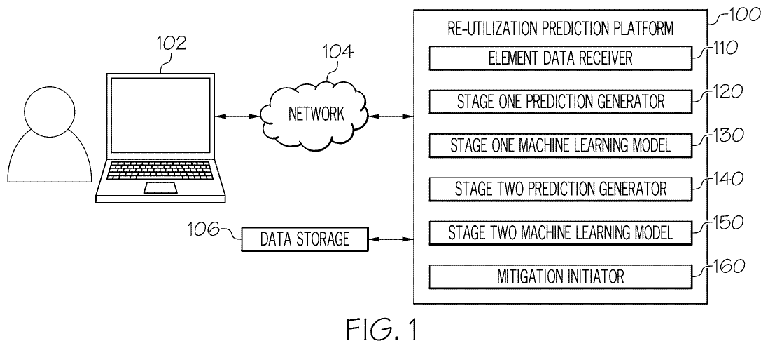

FIG. 1 depicts an example system infrastructure for a re-utilization prediction platform, according to one or more embodiments. As depicted in FIG. 1, client device 102 communicates with re-utilization prediction platform 100 over network 104. Data storage 106 communicates directly with re-utilization prediction platform 100 as shown in FIG. 1, or communicates with re-utilization prediction platform 100 over network 104. Re-utilization prediction platform 100 includes element data receiver 110, stage one prediction generator 120, stage one machine learning model 130, stage two prediction generator 140, stage two machine learning model 150, and mitigation initiator 160.

Element data receiver 110 receives data associated with an element or entity, such as a patient, for example, during a first stage, and receive different data during a second stage. Stage one prediction generator 120 generates a prediction for the element in a first stage, and uses stage one machine learning model 130. Stage two prediction generator 140 updates the prediction for the element in a second stage, and uses stage two machine learning model 150. Mitigation initiator 160 uses the updated prediction to initiate performance of a mitigation action.

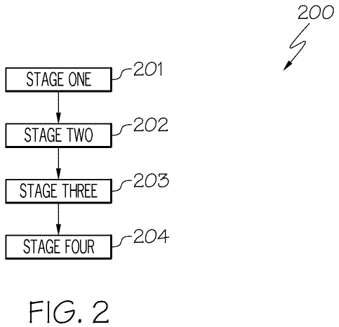

FIG. 2 depicts an example flowchart of a re-utilization prediction, according to one or more embodiments. As depicted in FIG. 2, re-utilization prediction 200 includes stage one prediction 201, stage two prediction 202, stage three prediction 203, and stage four prediction 204 (e.g., as examples of stage one prediction generator 120 and stage two prediction generator 140).

For example, the four different stages of utilization of the resource system by an entity include (1) initial utilization of the resource system by the entity as stage one prediction 201, (2) during utilization of the resource system by the entity as stage two prediction 202, (3) release of the entity from the resource system as stage three prediction 203, and (4) post-release of the entity from the resource system as stage four prediction 204. Additionally, re-utilization prediction 200 periodically determines high-risk entities during one or more of the four stages above. For example, high-risk entities are determined on a daily basis during utilization of the resource system by the entity. Datasets (e.g., features) used at one stage to determine a risk of a subsequent re-utilization of an entity can differ from datasets used at another stage.

In re-utilization prediction 200, a risk of a subsequent re-utilization of a resource system by an entity is determined using one or more machine learning models. For example, four machine learning models respectively corresponding to the four different stages of utilization (stage one prediction 201, stage two prediction 202, stage three prediction 203, and stage four prediction 204) are used, each taking into account a different set of datasets. For example, a machine learning model for a subsequent stage (e.g., stage four prediction 204) of the utilization of the resource system by the entity uses datasets associated with the subsequent stage (e.g., stage four prediction 204) along with datasets associated with one or more previous stages (e.g., stage one prediction 201, stage two prediction 202, stage three prediction 203).

One of the machine learning techniques that is useful and effective for the re-utilization prediction 200 is a neural network, which is a type of supervised machine learning. Nonetheless, other machine learning techniques and frameworks may be used to perform the methods contemplated by the present disclosure. For example, re-utilization prediction 200 may be realized using other types of supervised machine learning such as regression problems, random forest, etc., using unsupervised machine learning such as cluster algorithms, principal component analysis (PCA), etc., and/or using reinforcement learning.

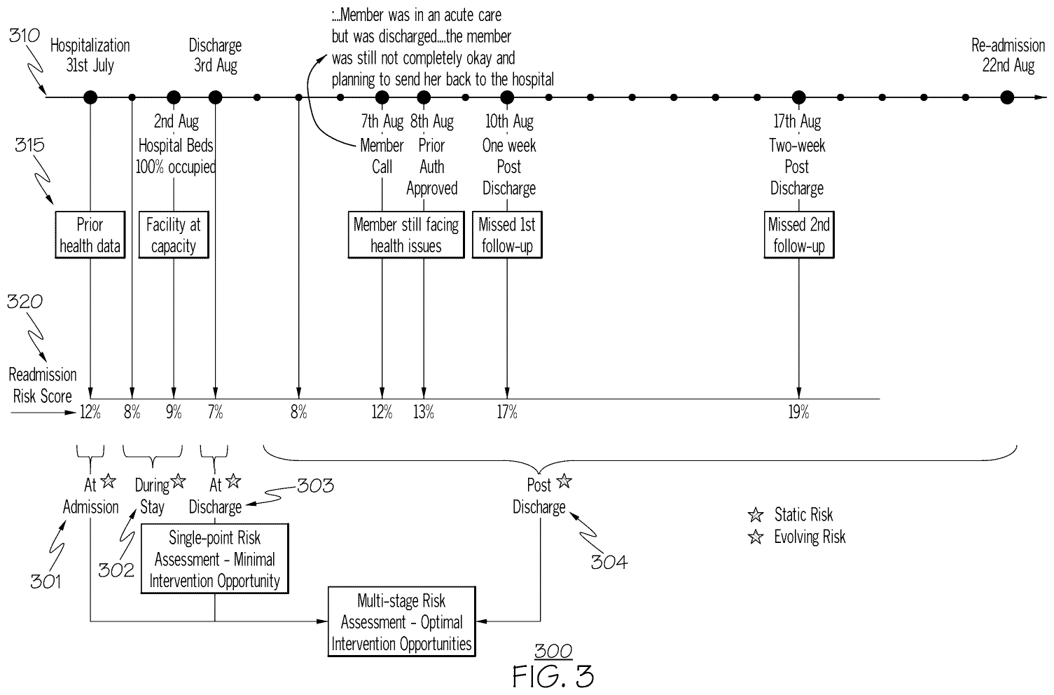

FIG. 3 depicts an example timeline of utilization of a resource system and corresponding re-utilization predictions, according to one or more embodiments. As depicted in data example 300 in FIG. 3, timeline 310 includes events occurring on various dates, data 315 includes data provided to a system such as re-utilization prediction platform 100, for example, and re-utilization predictions 320 include re-utilization predictions corresponding to the events in timeline 310. The re-utilization predictions are generated by stage one prediction 301, stage two prediction 302, stage three prediction 303, and stage four prediction 304 (e.g., as examples of stage one prediction 201, stage two prediction 202, stage three prediction 203, and stage four prediction 204.)

Re-utilization prediction platform 100 improves a readmission risk prediction paradigm from a single point in time assessment to a more holistic multi-stage risk prediction framework, such as multiple stages (e.g., stage one prediction 301, stage two prediction 302, stage three prediction 303, and stage four prediction 304) of a healthcare service episode of a member, which may be used to enable intervention opportunities. Re-utilization prediction platform 100 includes multiple AI/ML models across a member's healthcare journey, where features of the models are a function of respective stages of the journey. For example, the models include (i) one-time score models to score a static risk, such as at a time of admission and at a time of discharge, and (ii) daily (or periodic) score models to score an evolving risk, such as during hospitalization and post discharge.

At each stage, there are two different processes involved, and both include data processing and data cleaning. Re-utilization prediction platform 100 includes model development, validation, and performance testing. Re-utilization prediction platform 100 provides delivery of the high-risk members daily by scoring using ML models. Re-utilization prediction platform 100 initiates mitigation actions for timely intervention that can improve long-term health of members and further enhance member experience. Re-utilization prediction platform 100 provides a multistage risk prediction framework that enables a focus on high-risk members at each stage and leverages intervention opportunities that enable better health outcomes.

Re-utilization prediction platform 100 improves a readmission risk paradigm by identifying members with higher likelihood of hospital readmission at each stage of their healthcare service episode journey, to enable proactive interventions at the right time to reduce the readmission risk and improve overall health outcomes. There are multiple reasons for which likelihood of readmission can increase or decrease from the admission stage (e.g., stage one prediction 301) to post discharge stage (e.g., stage four prediction 304). Throughout a healthcare episode (e.g., July 31 through August 22 as depicted in FIG. 3), if a member has not been provided with required level of quality of care, a readmission risk can increase. For example, during hospital stay (e.g., stage two prediction 302), procedures should be performed with a quality to avoid readmission. Post-discharge (e.g., stage four prediction 304), if the patient doesn't keep up with their follow up appointments or isn't adhering to their medication schedule, there can be a higher risk of readmission.

Conventional solutions predict readmission risk at a time of discharge (e.g., August 3 as depicted in FIG. 3). However, the conventional models rely on members' recent discharge information and episodic data to predict readmission risk after the member has already been discharged, which means that potentially low quality care has already been given to the member. Therefore, conventional solutions provide a minimal intervention opportunity, and the scope of care plan recommendations is limited to the time post discharge. Re-utilization prediction platform 100 provides a multistage approach to identify the high-risk readmission members according to where they currently are in their episodic journey. This multistage approach provides timely prediction of readmission risk, and thus, enables proactive interventions at an earlier stage, to ensure that quality care and treatment can be provided from the admission onset (e.g., stage one prediction 301) to the discharge stage (e.g., stage four prediction 304) to reduce the risk of readmission.

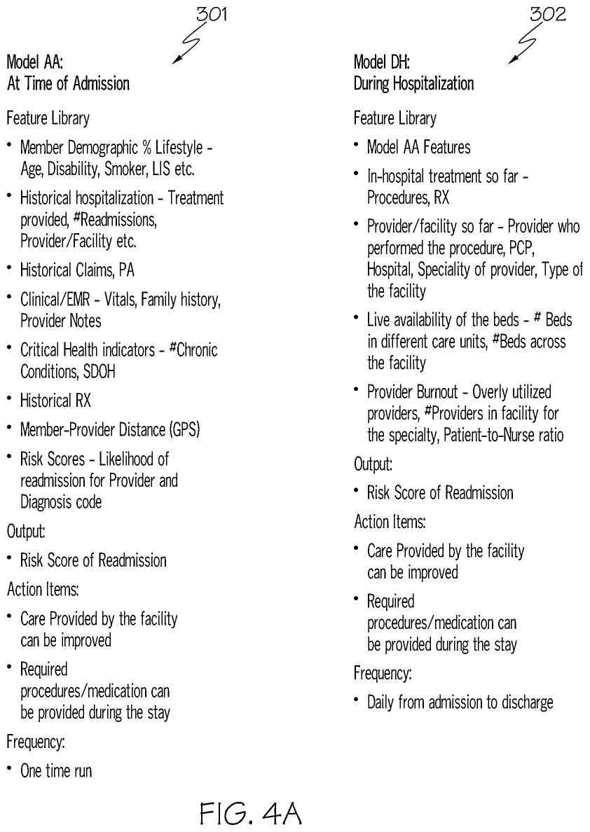

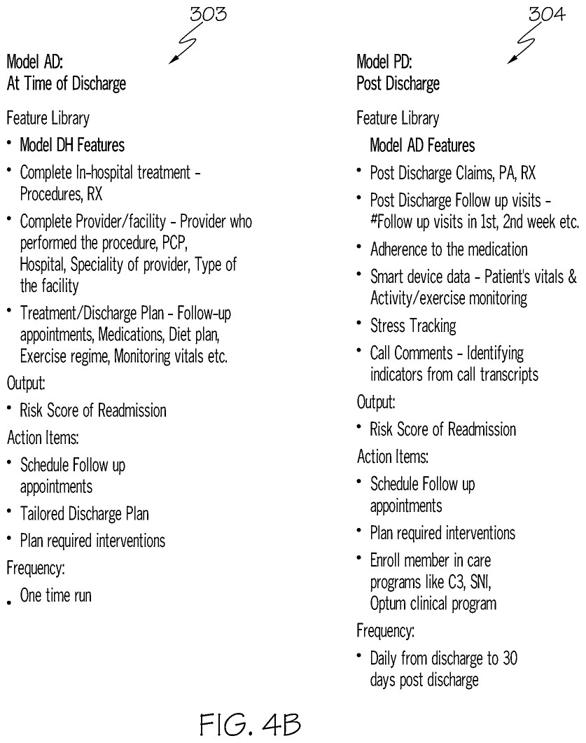

FIG. 4A and FIG. 4B depict an example system for re-utilization prediction using four trained machine-learning based models, according to one or more embodiments. As depicted in FIG. 4A and FIG. 4B, each of stage one prediction 301, stage two prediction 302, stage three prediction 303, and stage four prediction 304 includes a machine-learning based model. Each of stage one prediction 301, stage two prediction 302, stage three prediction 303, and stage four prediction 304 includes various features (e.g., input data), output data, mitigation actions, and operation frequency.

Re-utilization prediction platform 100 predicts a readmission risk across four different stages of the episode, from admission onset to post discharge, and thus, provides incremental benefits by enabling addition of valuable features which become available as the member moves from one stage to another. These features play a key role in enhancing the algorithm to determine the readmission risk more accurately and enable ML models to perform better as stages progress. For example, re-utilization prediction platform 100 provides an incremental readmission capture rate of approximately 5% to approximately 12% in the top risk percentiles, based on an aggregate high risk membership from at discharge and post discharge models, as compared to a capture rate from an “only at discharge” model. This capture rate is further enhanced by including the high-risk population from additional stage models. Re-utilization prediction platform 100 includes features in the models related to real time tracking of provider and/or facility metrics, as well as features related to members and their follow up interactions, to enhance model performance across all four stages.

A first stage model (e.g., stage one prediction 301) is configured for a time at admission (model AA). Model AA provides a prediction of readmission risk when the member is admitted to the hospital in the beginning of the health episode. Model AA receives data including member demographics, historical claims, historical prior authorizations, clinical and/or electronic medical records, distance between provider and member, chronic condition details of the member, risk scores for each provider, diagnostic code level, historical hospitalization-related features, prescription-related features, lifestyle features, and social determinants of health. Through risk identification, the initial care plan can be updated to ensure quality of care at the outset and the required procedures and/or medication can be provided with right quality and adherence during the stay itself, which reduces risk of readmission.

A second stage model (e.g., stage two prediction 302) is configured for a time during hospitalization (model DH). Model DH provides a prediction of readmission risk on a real time basis (e.g., daily risk scoring) during a member's stay in the hospital. At the second stage, in addition to data from model AA, model DH receives data including live availability of beds, provider burnout, partial in hospital treatment, and provider and facility related features of the member. Through risk identification, care provided by the facility can be assessed daily and further improved to address any gap in the initial care provided to the member and proactively enable the appropriate interventions, which reduces risk of readmission.

A third stage model (e.g., stage three prediction 303) is configured for a time at discharge (model AD). Model AD provides a prediction of readmission risk while the member is being discharged. At the third stage, in addition to data from model DH, model AD receives data including treatment and/or discharge plan and complete in hospital treatment and provider and facility related features of the member. The additional (complete) data increases the accuracy of model AD. Also, post identification, follow up appointments can be better scheduled, and discharge plans can be tailored, which reduces risk of readmission.

A fourth stage model (e.g., stage four prediction 304) is configured for a time post discharge (model PD). Model PD provides a prediction of readmission risk every day for approximately 30 days, for example, following the day the member is discharged. At the fourth stage, in addition to data from model AD, model PD receives data including post discharge features such as post-discharge follow-up visits, stress tracking, adherence to medication, claims, prior authorizations, provider and/or facility visits, call comments, patient's vitals, and patient's smart device related information. At the fourth stage, with additional data, model PD provides the most accurate prediction. However, the opportunity window is short as the member is already discharged and closer to readmission. Also, post identification, follow up appointments can be scheduled, and members can be contacted to ensure that they are keeping the appointments, and members can be enrolled in care programs such as disease specific clinical programs.

Multi-stage readmission risk prediction can be used to create appropriate intervention opportunities for personalized care for high-risk patients at each touchpoint of their health episode. Support care-providers can use multi-stage readmission risk prediction to create and/or modify the care plan and medications during the hospital stay and even identify the provider. Multi-stage readmission risk prediction can be used to decide if a patient is ready for discharge or should be considered for an intervention program, eventually reducing the number of readmissions and curbing healthcare cost. Multi-stage readmission risk prediction can be used to educate the patients around their health condition. Multi-stage readmission risk prediction can be used to create a discharge plan at time of discharge and make appointments for clinician follow-up and/or testing. Post discharge, multi-stage readmission risk prediction can be used to provide reinforcement of the plan and maintain communication in case the member fails to keep follow-ups or there is a deviation in vitals and/or lab results. Multi-stage readmission risk prediction can be used to review appropriate steps with members and/or caregivers to be taken in adverse situations.

Multi-stage readmission risk prediction can be leveraged to assess provider and regional performance and assist providers in identifying high-risk patients and populations in their practice and deliver recommendations to make required changes. When a member is admitted to the hospital for a certain health episode, the risk of readmission would be predicted either one-time or daily, based on the current stage of episode. This multistage identification also increases the accuracy of the readmission risk prediction as more data and/or features become available as stages progress, and added into the solution workflow to improve performance of the models.

For each stage, different data and/or features are available. At a time of admission (model AA), member demographics, historical claims, historical prior authorizations, clinical and/or electronic medical records, distance between provider and member, chronic conditions, risk scores for each provider, diagnostic code level, historical hospitalization-related features, prescription related features, lifestyle features, and social determinants of health features are available. During hospitalization (in addition to model AA data), live availability of beds, provider burnout, partial in-hospital treatment, and partial provider and facility related features are also available. During discharge (in addition to model DH data), complete in-hospital treatment, provider and/or facility and treatment and/or discharge plan related features are also available. During post-discharge (in addition to model AD data), post discharge features such as post-discharge follow-up visits, stress tracking, adherence to medication, claims, prior authorizations, provider and/or facility visits, call comments, patients vitals, and patient's smart device related features are also available.

The different data and/or features include various information for one or more of the first, second, third, or fourth stage models, for example. For example, admissions/discharges data and other relevant patient data includes procuring the admissions, discharges data along with relevant historical health and demographics data. This aids in understanding the condition of the member and identifying the specific features/markers which are an indication of readmission risk. The data includes identifying the first admission, discharge, and any re-admissions of the member to find the eligible population. The data includes demographic and lifestyle features such as age, disability indicator, smoker indicator, bankruptcy, living conditions, inactivity/time spent exercising weekly, married, type of job/income/primary earner/employed, education, alcohol intake.

The data includes prior authorization and post discharge features such as denied prior authorizations and accepted/valid prior authorizations. The data includes hospitalization features such as historical admissions, diagnosis condition, in-hospital complications, discharge composition (e.g., home, skilled nursing facility, home-health care service, rehabilitation, short-term), complaints (e.g., shortness of breath), weakness, respiratory, note from physician, medication details, physician, medication note, plan at time of discharge, mortality rate of the hospital.

The data includes historical claims and post discharge features such as a number of claims in diagnosis condition/procedure codes, cost related variables, participating and non-participating provider claims, denied and paid claims, and number of specialties. The data includes any chronic condition of the member, number of chronic conditions, health conditions (e.g., comorbidities, diabetes, obesity, and hypertension), history of depression, chronic obstructive pulmonary disease, number of high cost claims, procedures, renal disease, and risk adjustment factor. The data includes electronic medical records and clinical data such as family history, provider notes, weight, height, body mass index, blood pressure, A1C/glucose levels, lab results, and biometric data.

The data includes live availability of beds at the admission/readmission facility, which impacts the duration of the stay of the member. Early discharge can lead to readmissions. The data includes provider burnout, which can lead to degradation in the service provided, which can in turn increase readmission probability. The data includes a distance between provider and member. The shorter the distance, the easier the member may visit the provider. To mitigate longer distances, video follow ups can be scheduled instead of in person meeting for routine follow ups. The data includes tracking of smart device data. For example, adherence to diet/medicine/exercise can reduce readmission probability. The data includes stress tracking, as stress can be measured and impacts the readmission probability. The data includes call comments, such as call transcripts of members with providers.

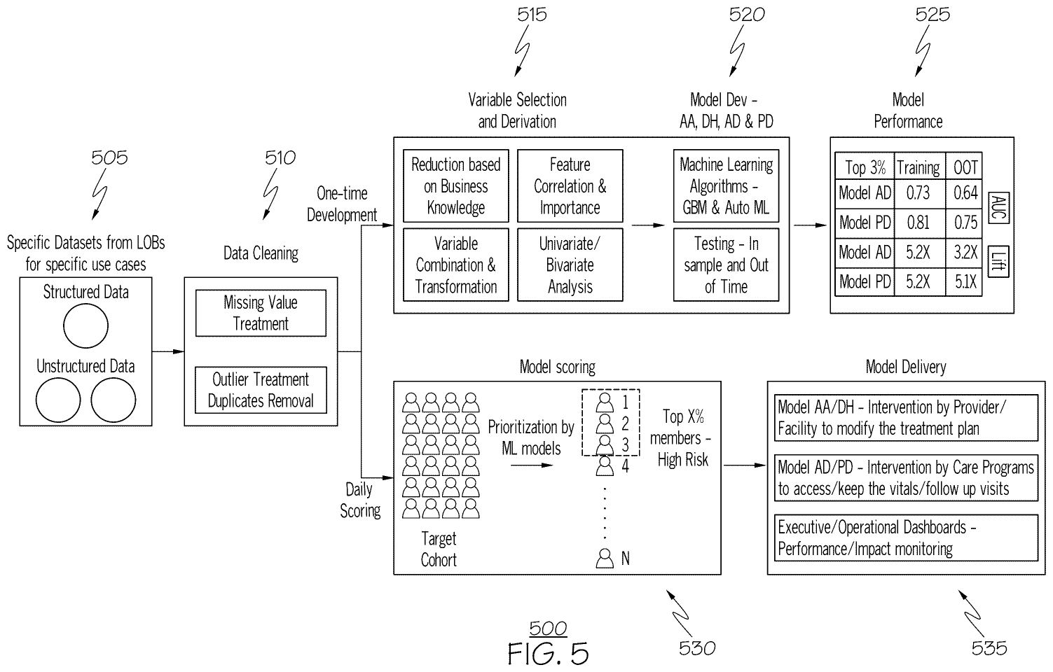

FIG. 5 depicts an example system infrastructure for a re-utilization prediction platform, according to one or more embodiments. As depicted in FIG. 5, system infrastructure 500 includes datasets 505, data cleaning 510, variable selection and derivation 515, model training 520, model testing 525, model scoring 530, and model delivery 535.

One or more embodiments provide feature engineering of all the various structured and/or unstructured data. Feature engineering includes performing data cleaning tasks such as missing value treatment, outlier detection and treatment, removing unwanted data such as duplicates or irrelevant information, and correcting structural errors, for example. Feature engineering includes creating and/or identifying features from existing dimensions using various techniques such as univariate/bivariate analysis, feature correlation, and feature importance, for example.

Re-utilization prediction platform 100 provides a four stage approach, with four different ML models built for the four respective stages, along with different feature sets. Model AA corresponds to a time of admission and runs at the day of admission to predict the readmission risk. Model DH corresponds to a time during hospitalization and runs when the member is admitted in the hospital for the first time until the day of discharge to predict the readmission risk. Model AD corresponds to a time of discharge and runs on the day of discharge to predict the readmission risk. Model PD corresponds to a time post discharge and runs daily post discharge until 30 days, for example, or until readmission, whichever is earlier, to predict the readmission risk

Re-utilization prediction platform 100 uses various classification ML algorithms and/or neural network models. For example, various classification algorithms such as logistic regression, random forest, gradient boosting machines, and neural networks are used to predict the readmission risk.

Re-utilization prediction platform 100 is deployed with a daily (or periodic) run of an ensemble model of eligible admissions and/or discharges for the day (or time segment). One or more embodiments determines the high-risk members from each model at each stage, and shares the output list with respective member care programs. For model AA and model DH, with collaboration with a provider and/or facility, a member is provided with required procedures and care. For model AD and model PD, the member is provided with a customized discharge plan, and provides an outreach in case the member is not following the program or in case a test result or smart device indicates a discrepancy and/or alert. One or more embodiments provide executive and operational dashboards that can be used to monitor the performance of the models and the impact of the models.

Re-utilization prediction platform 100 uses a distance between the provider/facility and member to determine readmission risk. For example, if a member is travelling from Alaska to Florida to get a heart surgery, the member will also need to visit their provider for follow-ups to monitor the vitals and health. However, because the distance between the member and provider is large, the member is more likely to miss the follow-up visits, which leads to higher readmission risk.

Re-utilization prediction platform 100 uses call transcripts to determine readmission risk. For example, during a call with healthcare agents, a patient might share information which can be an indication of health care delay or drop in quality of care, and thus, increasing the risk of readmission. For example, a transcript of the call includes “ . . . member was in an acute care but was discharged . . . the member was still not completely okay and planning to send her back to the hospital . . . ”.

Conventional solutions for readmission prediction do not consider the entire health service episode and instead sue a single point assessment. Re-utilization prediction platform 100 improves the overall readmission prediction framework by using a multistage approach, which is not a single point in time, but is spread across a healthcare service episode. The prediction is from admission onset to post discharge, which results in a higher capture rate of readmission cases, and enables opportunities for proactive interventions by early risk detection.

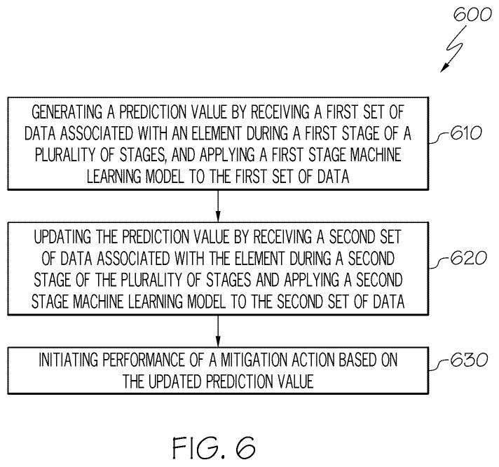

FIG. 6 depicts a flowchart of a method of re-utilization prediction using two trained machine-learning based models, according to one or more embodiments. As depicted in FIG. 6, method 600 includes various operations, and is performed by the re-utilization prediction platform 100 (or the components therein). Method 600 includes receiving, by one or more processors, a first set of data associated with an element during a first stage (e.g., stage one prediction 301) of a plurality of stages, and applying, by the one or more processors, a first stage machine learning model to the first set of data to generate a prediction value (operation 610). The first stage machine learning model is trained with a first set of feature data that is available during the first stage. The prediction value represents a likelihood of re-utilization of a resource system by the element.

Method 600 includes updating, by the one or more processors, the prediction value by: receiving a second set of data associated with the element during a second stage (e.g., stage two prediction 302) of the plurality of stages, and applying a second stage machine learning model to the second set of data (operation 620). One or more of the first stage machine learning model or the second stage machine learning model includes one or more of a neural network, regression, random forest, or gradient boosting. The updating, by the one or more processors, the prediction value is performed periodically. The second stage machine learning model is trained with (i) the first set of feature data that is available during the first stage, and (ii) a second set of feature data that is available during the second stage.

Method 600 includes initiating, by the one or more processors, performance of a mitigation action based on the updated prediction value (operation 630). The mitigation action includes one or more of updating a care plan for the element in a resource system, addressing a gap in care for the element in the resource system, generating a follow-up for the element, updating a discharge plan for the element, or contacting the element. The initiating, by the one or more processors, the performance of the mitigation action includes generating a display including one or more of the mitigation action or the updated prediction value.

Updating, by the one or more processors, the prediction value further includes receiving a third set of data associated with the element during a third stage of the plurality of stages; and applying a third stage machine learning model to the third set of data, wherein the third stage machine learning model is trained with (i) the first set of feature data that is available during the first stage, (ii) the second set of feature data that is available during the second stage, and (iii) a third set of feature data that is available during the third stage.

Updating, by the one or more processors, the prediction value further includes receiving a fourth set of data associated with the element during a fourth stage of the plurality of stages; and applying a fourth stage machine learning model to the fourth set of data, wherein the fourth stage machine learning model is trained with (i) the first set of feature data that is available during the first stage, (ii) the second set of feature data that is available during the second stage, (iii) the third set of feature data that is available during the third stage, and (iv) a fourth set of feature data that is available during the fourth stage.

For example, the first stage is a stage of initial utilization of a resource system by the element, the second stage is a stage during a stay of the element in the resource system, the third stage is a stage of discharge of the element from the resource system, and the fourth stage is a stage after discharge of the element from the resource system. The first set of data includes one or more of age of the element, hospitalizations, claims, biometric data, health indicators, or distance from an address of the element to a resource system. The second set of data includes one or more of resource system treatment or resource system information. The third set of data includes one or more of facility treatment, resource system information, facility information, or a discharge plan. The fourth set of data includes one or more of a claim or smart device monitoring data. One or more of the first set, the second set, the third set, or the fourth set of data may be static data (i.e. data that is retrieved and not updated) or may be dynamic data (i.e. data that is retrieved and later updated).

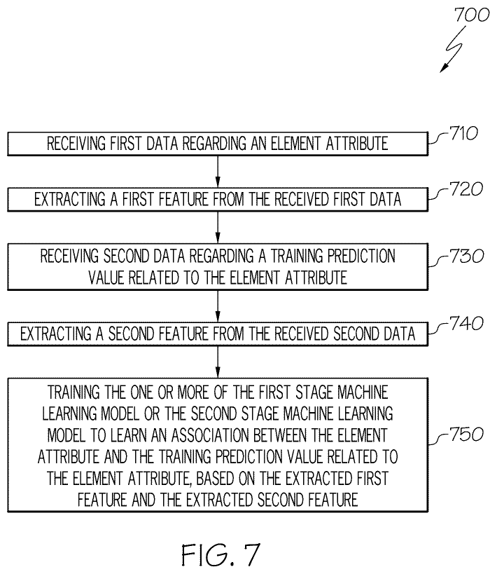

FIG. 7 depicts a flowchart of a method of training a machine-learning based model to determine a re-utilization prediction, according to one or more embodiments. The training is performed by one or more processors of the re-utilization prediction platform 100, or one or more processors of another server/device that is in communication with the re-utilization prediction platform 100 via network 104. As depicted in FIG. 7, method 700 includes various operations for training one or more of the first stage machine learning model (e.g., stage one prediction 301) or the second stage machine learning model (e.g., stage two prediction 302). Method 700 includes receiving first data regarding an element attribute (operation 710). Method 700 includes extracting a first feature from the received first data (operation 720). Method 700 includes receiving second data regarding a training prediction value related to the element attribute (operation 730). Method 700 includes extracting a second feature from the received second data (operation 740). Method 700 includes training the one or more of the first stage machine learning model or the second stage machine learning model to learn an association between the element attribute and the training prediction value related to the element attribute, based on the extracted first feature and the extracted second feature (operation 750).

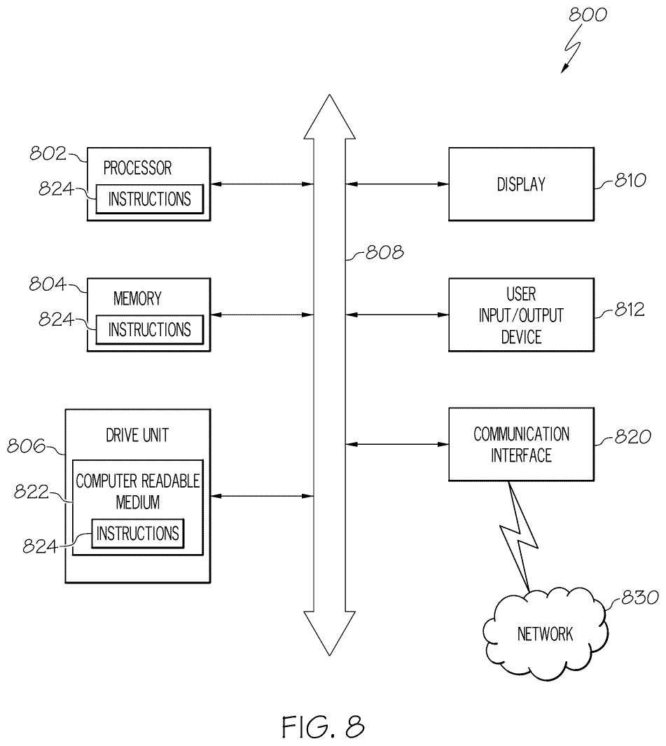

FIG. 8 depicts an implementation of a computer system that executes techniques presented herein, according to one or more embodiments. Computer system 800 can include a set of instructions that can be executed to cause the computer system 800 to perform any one or more of the methods or computer-based functions disclosed herein. The computer system 800 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,” or a “server” includes one or more processors.

In a networked deployment, the computer system 800 operates in the capacity of a server or as a client user computer in a server-client user network environment, or as a peer computer system in a peer-to-peer (or distributed) network environment. The computer system 800 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 800 can be implemented using electronic devices that provide voice, video, or data communication. Further, while the computer system 800 is illustrated as a single system, the term “system” shall also be taken to include any collection of systems or sub-systems that individually or jointly execute a set, or multiple sets, of instructions to perform one or more computer functions.

As illustrated in FIG. 8, the computer system 800 includes a processor 802, e.g., a central processing unit (CPU), a graphics processing unit (GPU), or both. The processor 802 can be a component in a variety of systems. For example, the processor 802 is part of a standard personal computer or a workstation. The processor 802 is one or more processors, digital signal processors, application specific integrated circuits, field programmable gate arrays, servers, networks, digital circuits, analog circuits, combinations thereof, or other now known or later developed devices for analyzing and processing data. The processor 802 implements a software program, such as code generated manually (e.g., programmed).

The computer system 800 includes a memory 804 that can communicate via a bus 808. The memory 804 is a main memory, a static memory, or a dynamic memory. The memory 804 includes, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media, and the like. In one implementation, the memory 804 includes a cache or random-access memory for the processor 802. In alternative implementations, the memory 804 is separate from the processor 802, such as a cache memory of a processor, the system memory, or other memory. The memory 804 can be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 804 is operable to store instructions executable by the processor 802. The functions, acts or tasks illustrated in the figures or described herein are performed by the processor 802 executing the instructions stored in the memory 804. The functions, acts or tasks are independent of the particular type of instructions set, storage media, processor or processing strategy and are performed by software, hardware, integrated circuits, firm-ware, micro-code and the like, operating alone or in combination. Likewise, processing strategies can include multiprocessing, multitasking, parallel processing, and the like.

As shown, the computer system 800 further included a display 810, such as a liquid crystal display (LCD), an organic light emitting diode (OLED), a flat panel display, a solid-state display, a cathode ray tube (CRT), a projector, a printer or other now known or later developed display device for outputting determined information. The display 810 acts as an interface for the user to see the functioning of the processor 802, or specifically as an interface with the software stored in the memory 804 or in a drive unit 806.

Additionally or alternatively, the computer system 800 includes an input/output device 812 configured to allow a user to interact with any of the components of the computer system 800. The input/output device 812 is a number pad, a keyboard, or a cursor control device, such as a mouse, or a joystick, touch screen display, remote control, or any other device operative to interact with the computer system 800.

The computer system 800 also or alternatively includes the drive unit 806 implemented as a disk or optical drive. The drive unit 806 includes a computer-readable medium 822 in which one or more sets of instructions 824, e.g., software, can be embedded. Further, the sets of instructions 824 embody one or more of the methods or logic as described herein. The instructions 824 reside completely or partially within the memory 804 and/or within the processor 802 during execution by the computer system 800. The memory 804 and the processor 802 can also include computer-readable media as discussed above.

In some systems, the computer-readable medium 822 includes the sets of instructions 824 or receives and executes the sets of instructions 824 responsive to a propagated signal so that a device connected to a network 830 can communicate voice, video, audio, images, or any other data over the network 830. Further, the sets of instructions 824 are transmitted or received over the network 830 via a communication port or interface 820, and/or using the bus 808. The communication port or interface 820 is a part of the processor 802 or is a separate component. The communication port or interface 820 is created in software or is a physical connection in hardware. The communication port or interface 820 are configured to connect with the network 830, external media, the display 810, or any other components in the computer system 800, or combinations thereof. The connection with the network 830 is a physical connection, such as a wired Ethernet connection or is established wirelessly as discussed below. Likewise, the additional connections with other components of the computer system 800 are physical connections or are established wirelessly. The network 830 is alternatively directly connected to the bus 808.

While the computer-readable medium 822 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that cause a computer system to perform any one or more of the methods or operations disclosed herein. In some examples, the computer-readable medium 822 is non-transitory, and is tangible.

The computer-readable medium 822 can include a solid-state memory such as a memory card or other package that houses one or more non-volatile read-only memories. The computer-readable medium 822 can be a random-access memory or other volatile re-writable memory. Additionally or alternatively, the computer-readable medium 822 can include a magneto-optical or optical medium, such as a disk or tapes or other storage device to capture carrier wave signals such as a signal communicated over a transmission medium. A digital file attachment to an e-mail or other self-contained information archive or set of archives are considered a distribution medium that is a tangible storage medium. Accordingly, the disclosure is considered to include any one or more of a computer-readable medium or a distribution medium and other equivalents and successor media, in which data or instructions are storable.

In an alternative implementation, dedicated hardware implementations, such as application specific integrated circuits, programmable logic arrays and other hardware devices, can be constructed to implement one or more of the methods described herein. Applications that include the apparatus and systems of various implementations can broadly include a variety of electronic and computer systems. One or more implementations described herein implement functions using two or more specific interconnected hardware modules or devices with related control and data signals that can be communicated between and through the modules, or as portions of an application-specific integrated circuit. Accordingly, the present system encompasses software, firmware, and hardware implementations.

The computer system 800 is connected to the network 830. The network 830 defines one or more networks including wired or wireless networks, such as the network 104 described in FIG. 1. The wireless network can be a cellular telephone network, an 802.11, 802.18, 802.20, or WiMAX network. Further, such networks include a public network, such as the Internet, a private network, such as an intranet, or combinations thereof, and utilize a variety of networking protocols now available or later developed including, but not limited to TCP/IP based networking protocols. The network 830 can include wide area networks (WAN), such as the Internet, local area networks (LAN), campus area networks, metropolitan area networks, a direct connection such as through a Universal Serial Bus (USB) port, or any other networks that allow for data communication. The network 830 is configured to couple one computing device to another computing device to enable communication of data between the devices. The network 830 generally is enabled to employ any form of machine-readable media for communicating information from one device to another. The network 830 includes communication methods by which information travels between computing devices. The network 830 can be divided into sub-networks. The sub-networks allow access to all of the other components connected thereto or the sub-networks restrict access between the components. The network 830 can be regarded as a public or private network connection and can include, for example, a virtual private network or an encryption or other security mechanism employed over the public Internet, or the like.

It will be understood that the steps of methods discussed are performed in one embodiment by an appropriate processor (or processors) of a processing (e.g., computer) system executing instructions (computer-readable code) stored in storage. It will also be understood that the disclosure is not limited to any particular implementation or programming technique and that the disclosure is implementable using any appropriate techniques for implementing the functionality described herein. The disclosure is not limited to any particular programming language or operating system.

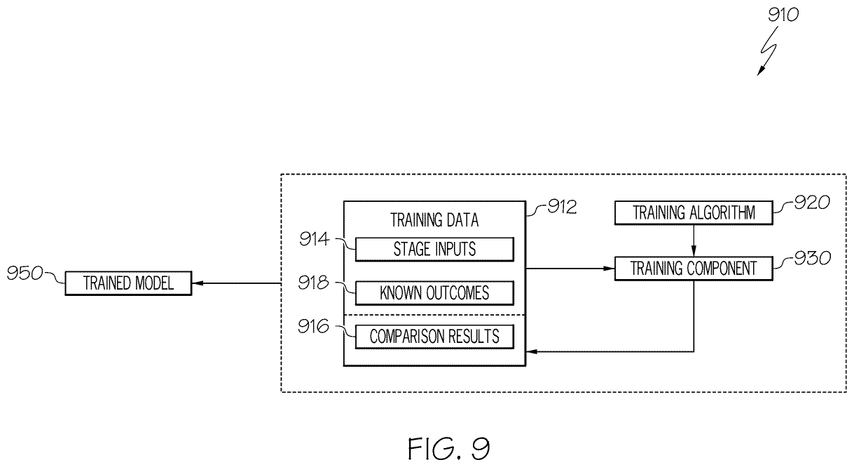

FIG. 9 depicts a flow diagram for training a machine learning model, according to one or more embodiments. As disclosed herein, one or more implementations disclosed herein may be applied by using a machine learning model. A machine learning model as disclosed herein may be trained using one or more components or operations of FIGS. 1-7. As shown in flow diagram 910 of FIG. 9, training data 912 may include one or more of stage inputs 914 and known outcomes 918 related to a machine learning model to be trained. The stage inputs 914 may be from any applicable source including a component or set shown in the figures provided herein.

The known outcomes 918 may be included for machine learning models generated based on supervised or semi-supervised training. An unsupervised machine learning model might not be trained using known outcomes 918. Known outcomes 918 may include known or desired outputs for future inputs similar to or in the same category as stage inputs 914 that do not have corresponding known outputs. The training data 912 and a training algorithm 920 may be provided to a training component 930 that may apply the training data 912 to the training algorithm 920 to generate a trained machine learning model 950. According to an implementation, the training component 930 may be provided comparison results 916 that compare a previous output of the corresponding machine learning model to apply the previous result to re-train the machine learning model. The comparison results 916 may be used by the training component 930 to update the corresponding machine learning model.

The training algorithm 920 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like.

A machine learning model disclosed herein may be trained by adjusting one or more weights, layers, and/or biases during a training phase. During the training phase, historical or simulated data may be provided as inputs to the model. The model may adjust one or more of its weights, layers, and/or biases based on such historical or simulated information. The adjusted weights, layers, and/or biases may be configured in a production version of the machine learning model (e.g., a trained model) based on the training. Once trained, the machine learning model may output machine learning model outputs in accordance with the subject matter disclosed herein. According to an implementation, one or more machine learning models disclosed herein continuously updates based on feedback associated with use or implementation of the machine learning model outputs.

It should be appreciated that in the above description of example embodiments of the present disclosure, various features of the present disclosure are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed embodiment requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the embodiments.

In the description provided herein, numerous specific details are set forth. However, it is understood that embodiments of the present disclosure can be practiced without these specific details. In other instances, well-known methods, structures, and techniques have not been shown in detail in order not to obscure an understanding of this description.

Thus, while there has been described what are believed to be the preferred embodiments of the present disclosure, those skilled in the art will recognize that other and further modifications can be made thereto without departing from the spirit of the embodiments, and it is intended to claim all such changes and modifications as falling within the scope of the embodiments. For example, any formulas given above are merely representative of procedures that can be used. Functionality can be added or deleted from the block diagrams and operations are interchangeable among functional blocks. Steps can be added or deleted to methods described within the scope of the present disclosure.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

The present disclosure further relates to the following aspects.

Example 1. A computer-implemented method comprising: receiving, by one or more processors, a first set of data associated with an element during a first stage of a plurality of stages; applying, by the one or more processors, a first stage machine learning model to the first set of data to generate a prediction value, wherein the first stage machine learning model is trained with a first set of feature data that is available during the first stage; updating, by the one or more processors, the prediction value by: receiving a second set of data associated with the element during a second stage of the plurality of stages; and applying a second stage machine learning model to the second set of data, wherein the second stage machine learning model is trained with (i) the first set of feature data that is available during the first stage, and (ii) a second set of feature data that is available during the second stage; and initiating, by the one or more processors, performance of a mitigation action based on the updated prediction value.

Example 2. The computer-implemented method of any of the previous examples, further comprising: training one or more of the first stage machine learning model or the second stage machine learning model by: receiving first data regarding an element attribute; extracting a first feature from the received first data; receiving second data regarding a training prediction value related to the element attribute; extracting a second feature from the received second data; and training the one or more of the first stage machine learning model or the second stage machine learning model to learn an association between the element attribute and the training prediction value related to the element attribute, based on the extracted first feature and the extracted second feature.

Example 3. The computer-implemented method of any of the previous examples, wherein one or more of the first stage machine learning model or the second stage machine learning model includes one or more of a neural network, regression, random forest, or gradient boosting.

Example 4. The computer-implemented method of any of the previous examples, wherein the updating, by the one or more processors, the prediction value further comprises: receiving a third set of data associated with the element during a third stage of the plurality of stages; and applying a third stage machine learning model to the third set of data, wherein the third stage machine learning model is trained with (i) the first set of feature data that is available during the first stage, (ii) the second set of feature data that is available during the second stage, and (iii) a third set of feature data that is available during the third stage.

Example 5. The computer-implemented method of example 4, wherein the updating, by the one or more processors, the prediction value further comprises: receiving a fourth set of data associated with the element during a fourth stage of the plurality of stages; and applying a fourth stage machine learning model to the fourth set of data, wherein the fourth stage machine learning model is trained with (i) the first set of feature data that is available during the first stage, (ii) the second set of feature data that is available during the second stage, (iii) the third set of feature data that is available during the third stage, and (iv) a fourth set of feature data that is available during the fourth stage.

Example 6. The computer-implemented method of example 5, wherein: the first stage is a stage of initial utilization of a resource system by the element, the second stage is a stage during a stay of the element in the resource system, the third stage is a stage of discharge of the element from the resource system, and the fourth stage is a stage after discharge of the element from the resource system.

Example 7. The computer-implemented method of any of the previous examples, wherein the first set of data includes one or more of element demographics, historical claims, historical prior authorizations, clinical and/or electronic medical records, distance between resource system and element, chronic condition details of the element, risk scores for each resource system, diagnostic code level, historical hospitalization-related features, prescription-related features, lifestyle features, or social determinants of health.

Example 8. The computer-implemented method of any of the previous examples, wherein the second set of data includes one or more of live availability of beds, resource system burnout, partial in hospital treatment, or resource system and facility related features of the element.

Example 9. The computer-implemented method of example 4, wherein the third set of data includes one or more of facility treatment, resource system information, facility information, or a discharge plan.

Example 10. The computer-implemented method of example 5, wherein the fourth set of data includes one or more of post-discharge follow-up visits, stress tracking, adherence to medication, claims, prior authorizations, resource system and/or facility visits, call comments, vitals of the element, or information related to a smart device of the element.

Example 11. The computer-implemented method of any of the previous examples, wherein the mitigation action includes one or more of updating a care plan for the element in a resource system, addressing a gap in care for the element in the resource system, generating a follow-up for the element, updating a discharge plan for the element, or contacting the element.

Example 12. The computer-implemented method of any of the previous examples, wherein the updating, by the one or more processors, the prediction value is performed periodically.

Example 13. The computer-implemented method of any of the previous examples, wherein the prediction value represents a likelihood of re-utilization of a resource system by the element.

Example 14. The computer-implemented method of any of the previous examples, wherein the initiating, by the one or more processors, the performance of the mitigation action includes generating a display including one or more of the mitigation action or the updated prediction value.

Example 15. A system comprising: one or more processors; and one or more memories storing instructions that, when executed by the one or more processors, cause the one or more processors to perform operations including: receiving a first set of data associated with an element during a first stage of a plurality of stages; applying a first stage machine learning model to the first set of data to generate a prediction value, wherein the first stage machine learning model is trained with a first set of feature data that is available during the first stage; updating the prediction value by: receiving a second set of data associated with the element during a second stage of the plurality of stages; and applying a second stage machine learning model to the second set of data, wherein the second stage machine learning model is trained with (i) the first set of feature data that is available during the first stage, and (ii) a second set of feature data that is available during the second stage; and initiating performance of a mitigation action based on the updated prediction value.

Example 16. The system of example 15, wherein the operations further include: training one or more of the first stage machine learning model or the second stage machine learning model by: receiving first data regarding an element attribute;

extracting a first feature from the received first data; receiving second data regarding a training prediction value related to the element attribute; extracting a second feature from the received second data; and training the one or more of the first stage machine learning model or the second stage machine learning model to learn an association between the element attribute and the training prediction value related to the element attribute, based on the extracted first feature and the extracted second feature.

Example 17. The system of any of examples 15-16, wherein one or more of the first stage machine learning model or the second stage machine learning model includes one or more of a neural network, regression, random forest, or gradient boosting.

Example 18. The system of any of examples 15-17, wherein the updating the prediction value further comprises: receiving a third set of data associated with the element during a third stage of the plurality of stages; applying a third stage machine learning model to the third set of data, wherein the third stage machine learning model is trained with (i) the first set of feature data that is available during the first stage, (ii) the second set of feature data that is available during the second stage, and (iii) a third set of feature data that is available during the third stage; receiving a fourth set of data associated with the element during a fourth stage of the plurality of stages; and applying a fourth stage machine learning model to the fourth set of data, wherein the fourth stage machine learning model is trained with (i) the first set of feature data that is available during the first stage, (ii) the second set of feature data that is available during the second stage, (iii) the third set of feature data that is available during the third stage, and (iv) a fourth set of feature data that is available during the fourth stage.

Example 19. The system of example 18, wherein: the first stage is a stage of initial utilization of a resource system by the element, the second stage is a stage during a stay of the element in the resource system, the third stage is a stage of discharge of the element from the resource system, and the fourth stage is a stage after discharge of the element from the resource system.

Example 20. A non-transitory computer readable medium storing instructions that, when executed by one or more processors, cause the one or more processors to perform operations comprising: receiving a first set of data associated with an element during a first stage of a plurality of stages; applying a first stage machine learning model to the first set of data to generate a prediction value, wherein the first stage machine learning model is trained with a first set of feature data that is available during the first stage; updating the prediction value by: receiving a second set of data associated with the element during a second stage of the plurality of stages; and applying a second stage machine learning model to the second set of data, wherein the second stage machine learning model is trained with (i) the first set of feature data that is available during the first stage, and (ii) a second set of feature data that is available during the second stage; and initiating performance of a mitigation action based on the updated prediction value.

Example 21. The computer-implemented method of example 2, wherein the training is performed by the one or more processors.

Example 22. The computer-implemented method of example 2, wherein: the one or more processors are included in a first computing entity; and the training is performed by one or more processors included in a second computing entity.

Other embodiments of the disclosure will be apparent to those skilled in the art from consideration of the specification and practice of the invention disclosed herein. It is intended that the specification and examples be considered as example only, with a true scope and spirit of the invention being indicated by the following claims.

What is claimed is:

1. A computer-implemented method comprising:
receiving, by one or more processors, a first set of data associated with an element during a first stage of a plurality of stages;
applying, by the one or more processors, a first stage machine learning model to the first set of data to generate a prediction value, wherein the first stage machine learning model is trained with a first set of feature data that is available during the first stage;
updating, by the one or more processors, the prediction value by:
receiving a second set of data associated with the element during a second stage of the plurality of stages; and
applying a second stage machine learning model to the second set of data, wherein the second stage machine learning model is trained with (i) the first set of feature data that is available during the first stage, and (ii) a second set of feature data that is available during the second stage; and
initiating, by the one or more processors, performance of a mitigation action based on the updated prediction value.

2. The computer-implemented method of claim 1, further comprising:
training one or more of the first stage machine learning model or the second stage machine learning model by:
receiving first data regarding an element attribute;
extracting a first feature from the received first data;
receiving second data regarding a training prediction value related to the element attribute;
extracting a second feature from the received second data; and
training the one or more of the first stage machine learning model or the second stage machine learning model to learn an association between the element attribute and the training prediction value related to the element attribute, based on the extracted first feature and the extracted second feature.

3. The computer-implemented method of claim 1, wherein one or more of the first stage machine learning model or the second stage machine learning model includes one or more of a neural network, regression, random forest, or gradient boosting.

4. The computer-implemented method of claim 1, wherein the updating, by the one or more processors, the prediction value further comprises:
receiving a third set of data associated with the element during a third stage of the plurality of stages; and
applying a third stage machine learning model to the third set of data, wherein the third stage machine learning model is trained with (i) the first set of feature data that is available during the first stage, (ii) the second set of feature data that is available during the second stage, and (iii) a third set of feature data that is available during the third stage.

5. The computer-implemented method of claim 4, wherein the updating, by the one or more processors, the prediction value further comprises:
receiving a fourth set of data associated with the element during a fourth stage of the plurality of stages; and
applying a fourth stage machine learning model to the fourth set of data, wherein the fourth stage machine learning model is trained with (i) the first set of feature data that is available during the first stage, (ii) the second set of feature data that is available during the second stage, (iii) the third set of feature data that is available during the third stage, and (iv) a fourth set of feature data that is available during the fourth stage.

6. The computer-implemented method of claim 5, wherein:
the first stage is a stage of initial utilization of a resource system by the element,
the second stage is a stage during a stay of the element in the resource system,
the third stage is a stage of discharge of the element from the resource system, and
the fourth stage is a stage after discharge of the element from the resource system.

7. The computer-implemented method of claim 1, wherein the first set of data includes one or more of element demographics, historical claims, historical prior authorizations, clinical and/or electronic medical records, distance between resource system and element, chronic condition details of the element, risk scores for each resource system, diagnostic code level, historical hospitalization-related features, prescription-related features, lifestyle features, or social determinants of health.

8. The computer-implemented method of claim 1, wherein the second set of data includes one or more of live availability of beds, resource system burnout, partial in hospital treatment, or resource system and facility related features of the element.

9. The computer-implemented method of claim 4, wherein the third set of data includes one or more of facility treatment, resource system information, facility information, or a discharge plan.

10. The computer-implemented method of claim 5, wherein the fourth set of data includes one or more of post-discharge follow-up visits, stress tracking, adherence to medication, claims, prior authorizations, resource system and/or facility visits, call comments, vitals of the element, or information related to a smart device of the element.

11. The computer-implemented method of claim 1, wherein the mitigation action includes one or more of updating a care plan for the element in a resource system, addressing a gap in care for the element in the resource system, generating a follow-up for the element, updating a discharge plan for the element, or contacting the element.

12. The computer-implemented method of claim 1, wherein the updating, by the one or more processors, the prediction value is performed periodically.

13. The computer-implemented method of claim 1, wherein the prediction value represents a likelihood of re-utilization of a resource system by the element.

14. The computer-implemented method of claim 1, wherein the initiating, by the one or more processors, the performance of the mitigation action includes generating a display including one or more of the mitigation action or the updated prediction value.

15. A system comprising:
one or more processors; and
one or more memories storing instructions that, when executed by the one or more processors, cause the one or more processors to perform operations including:
receiving a first set of data associated with an element during a first stage of a plurality of stages;
applying a first stage machine learning model to the first set of data to generate a prediction value, wherein the first stage machine learning model is trained with a first set of feature data that is available during the first stage;
updating the prediction value by:
receiving a second set of data associated with the element during a second stage of the plurality of stages; and
applying a second stage machine learning model to the second set of data, wherein the second stage machine learning model is trained with (i) the first set of feature data that is available during the first stage, and (ii) a second set of feature data that is available during the second stage; and
initiating performance of a mitigation action based on the updated prediction value.

16. The system of claim 15, wherein the operations further include:
training one or more of the first stage machine learning model or the second stage machine learning model by:
receiving first data regarding an element attribute;
extracting a first feature from the received first data;
receiving second data regarding a training prediction value related to the element attribute;
extracting a second feature from the received second data; and
training the one or more of the first stage machine learning model or the second stage machine learning model to learn an association between the element attribute and the training prediction value related to the element attribute, based on the extracted first feature and the extracted second feature.

17. The system of claim 15, wherein one or more of the first stage machine learning model or the second stage machine learning model includes one or more of a neural network, regression, random forest, or gradient boosting.

18. The system of claim 15, wherein the updating the prediction value further comprises:
receiving a third set of data associated with the element during a third stage of the plurality of stages;
applying a third stage machine learning model to the third set of data, wherein the third stage machine learning model is trained with (i) the first set of feature data that is available during the first stage, (ii) the second set of feature data that is available during the second stage, and (iii) a third set of feature data that is available during the third stage;
receiving a fourth set of data associated with the element during a fourth stage of the plurality of stages; and
applying a fourth stage machine learning model to the fourth set of data, wherein the fourth stage machine learning model is trained with (i) the first set of feature data that is available during the first stage, (ii) the second set of feature data that is available during the second stage, (iii) the third set of feature data that is available during the third stage, and (iv) a fourth set of feature data that is available during the fourth stage.

19. The system of claim 18, wherein:
the first stage is a stage of initial utilization of a resource system by the element,
the second stage is a stage during a stay of the element in the resource system,
the third stage is a stage of discharge of the element from the resource system, and
the fourth stage is a stage after discharge of the element from the resource system.

20. A non-transitory computer readable medium storing instructions that, when executed by one or more processors, cause the one or more processors to perform operations comprising:
receiving a first set of data associated with an element during a first stage of a plurality of stages;
applying a first stage machine learning model to the first set of data to generate a prediction value, wherein the first stage machine learning model is trained with a first set of feature data that is available during the first stage;
updating the prediction value by:
receiving a second set of data associated with the element during a second stage of the plurality of stages; and
applying a second stage machine learning model to the second set of data, wherein the second stage machine learning model is trained with (i) the first set of feature data that is available during the first stage, and (ii) a second set of feature data that is available during the second stage; and
initiating performance of a mitigation action based on the updated prediction value.
